(19) United States
(12) Patent Application Publication
(10) Pub. No.: US 2010/0029987 A1
(43) Pub. Date: Feb. 4, 2010

(54) Crystalline Form of Rasagiline and Process for the Preparation Thereof

(75) Inventors: Pietro ALLEGRINI, San Donato Milanese (MI) (IT); Bruno Gaetano Romano', Casatenovo (LC) (IT); Emanuele Attolino, Palagiano (TA) (IT); Marco Artico, Parabiago (MI) (IT); Davide Rossi, Malgesso (VA) (IT)

Correspondence Address: ROTHWELL, FIGG, ERNST & MANBECK, P.C., 1425 K STREET, N.W., SUITE 800, WASHINGTON, DC, 20005, US

(73) Assignee: DIPHARMA FRANCIS S.r.I., Baranzate (MI) (IT)

(21) Appl. No.: 12/490,643

(22) Filed: Jun. 24, 2009

(30) Foreign Application Priority Data
Jul. 29, 2008 (IT) MI2008A001406
Jan. 22, 2009 (IT) MI2009A000067

(51) Int. Cl. C07C 211/30

(52) U.S. Cl. 564/308

(57) ABSTRACT

A process for the preparation of (R)—N-propargyl-1-aminoindane, or a salt thereof, comprising reacting 1-indanone with propargylamine, in presence of a mixture of sodium borohydride and acetic acid, to obtain N-propargyl-1-aminoindane; and its conversion into (R)—N-propargyl-1-aminoindane or a salt thereof.

### FIELD OF THE INVENTION

The present invention refers to a process for the preparation of (R)—N-propargyl-1-aminoindane or a salt thereof, in particular the mesylate salt.

### BACKGROUND OF THE ART

(R)—N-propargyl-1-aminoindane, commonly known as Rasagiline, having formula (I)

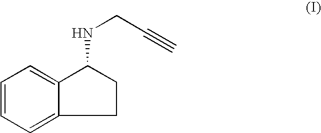

is known from U.S. Pat. No. 5,532,415, and is used in therapy as mesylate salt for the treatment of neurological diseases in particular for the treatment of Parkinson's disease. U.S. Pat. No. 5,532,415 discloses several methods for the preparation of Rasagiline and the mesylate salt thereof. In particular, Rasagiline can be directly prepared from (R)-1-aminoindane enantiomer by reaction with propargyl bromide or chloride or with a propargyl sulphonate (mesylate or tosylate) in the presence of an organic or inorganic base and optionally of a solvent.

U.S. Pat. No. 7,375,249 discloses the preparation of Rasagiline by hydrogenation of 1-indanone in the presence of an optically activated catalyst to obtain the corresponding (S)-indanol, followed by the derivatization of the hydroxyl group in a suitable leaving group, and the reaction with propargylamine.

Such process has several drawbacks, for example the high number of synthetic steps and the great formation of by-products which renders it unsuitable for the industrial scale production.

Therefore, there is the need of an alternative process for the preparation of Rasagiline, or a salt thereof, which is more suitable for industrial scale production.

### SUMMARY OF INVENTION

The invention provides a process for the preparation of N-propargylamino-1-indane and (R)—N-propargylamino-1-indane (Rasagiline) or a salt thereof, in particular the mesylate salt, which better satisfies the requirements for their production on industrial scale. A further object of the invention is to provide a process for the preparation of (R)—N-propargylamino-1-indane in solid crystalline form.

### BRIEF DISCLOSURE OF THE FIGURES AND ANALYTICAL METHODS

The crystalline forms of (R)—N-propargylamino-1-indane and of (R)—N-propargyl-1-aminoindane mesylate were characterized by X-Ray Powder Diffraction (XRPD), ¹H-NMR nuclear magnetic resonance spectrometry and Differential Scanning Calorimetry (DSC).

The water content of (R)—N-propargylamino-1-indane was determined by titration according to Karl-Fischer.

X-ray diffraction spectrum (XRPD) was recorded with an APD-2000 automatic diffractometer θ/θ for powders and liquids manufactured by Ital-Structures, under the following operative conditions: CuKα radiation (λ=1.5418 Å), scansion with angular interval 3-40° in 2θ with angular step of 0.03° for 1 sec.

DSC thermograms were recorded with the differential scansion calorimeter Mettler-Toledo DSC 822e, under the following operative conditions: aluminum capsules, 30-400° C. interval at the rate of 10° C./min, with nitrogen as purging gas (80 ml/min).

The particle size was determined with the known laser light scattering technique using a Malvern Mastersizer MS1 instrumentation under the following operative conditions:
300RF mm lens, with 2.4 mm laser beam length; and
500 mg sample dispersed in 10 ml of hexane (ACS reagent) with 1% SPAN 85®, no presonication, 2500 rpm stirring rate.

FIG. 1: XRPD spectrum of (R)—N-propargyl-1-aminoindane mesylate (crystalline Rasagiline mesylate);

FIG. 2: DSC thermogram of (R)—N-propargyl-1-aminoindane mesylate (crystalline Rasagiline mesylate),

FIG. 3: XRPD spectrum of (R)—N-propargyl-1-aminoindane (crystalline Rasagiline free base);

FIG. 4: DSC thermogram of (R)—N-propargyl-1-aminoindane (crystalline Rasagiline free base).

### DETAILED DESCRIPTION OF THE INVENTION

Object of this invention is a process for the preparation of N-propargyl-1-aminoindane, comprising reacting 1-indanone, having formula (II)

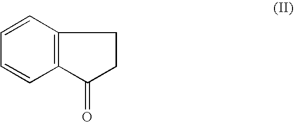

with propargylamine, in the presence of a mixture of sodium borohydride and acetic acid.

Acetic acid is preferably a concentrated acetic acid, in particular glacial acetic acid.

Preferably, the reaction is carried out in the presence of a solvent which can be an aromatic hydrocarbon such as toluene, or xylene, preferably toluene; or a cyclic or acyclic ether such as diethyl ether, diisopropylether, t-butyl methylether, dimethoxyethane, or tetrahydrofuran, more preferably tetrahydrofuran.

1-indanone can be added to the sodium borohydride and acetic acid mixture at a temperature comprised between about 10 and 40° C., preferably at about 30° C.

Propargylamine can be added to the reaction mixture by slow dropping, for example in about 5 hours, more preferably in about three hours, at a temperature comprised between 10 and 40° C., preferably at about 30° C. The reaction mixture is then preferably maintained at such temperature under stirring, for example for about 20 hours.

The so obtained reaction mixture can be then submitted to a “work up”, which can comprise, for example:

a) adding water to the reaction mixture, subsequently adding a base or an inorganic basic salt of an alkaline metal, and heating the mixture; and

b) separating the aqueous phase from the organic phase, concentrating the organic phase, isolating and recovering the product.

The base of an alkaline metal can be for example sodium hydroxide or potassium hydroxide; a basic inorganic salt of an alkaline metal can be for example potassium carbonate or sodium carbonate, potassium bicarbonate or sodium bicarbonate, preferably potassium carbonate. The addition of said basic agent to the reaction mixture is preferably in such a quantity so as to maintain the pH comprised between about 7 and 8.

The heating of the reaction mixture can be carried out at a temperature comprised between about 40 and 70° C., preferably at about 60-65° C.

The separation and the concentration to residue of the organic phase can be carried out according to known methods, for example by evaporation of the solvent under vacuum.

If desired, N-propargyl-1-aminoindane can be converted into a salt thereof. Such conversion can be carried out through a process comprising:

a′) resolving N-propargyl-1-aminoindane in (R)—N-propargyl-1-aminoindane tartrate; and

b′) converting it into free (R)—N-propargyl-1-aminoindane or in a different salt thereof.

The resolution of N-propargyl-1-aminoindane with L-(+)-tartaric acid can be carried out according to known methods, for example in presence of a C₁-C₄ alkanol, preferably methanol or ethanol.

The conversion of (R)—N-propargyl-1-aminoindane tartrate into (R)—N-propargyl-1-aminoindane or a salt thereof can be carried out according to known methods. Accordingly, for example (R)—N-propargyl-1-aminoindane can be obtained by using an aqueous solution of a base or of a basic salt of an inorganic acid with an alkaline metal, such as sodium or potassium hydroxide, sodium or potassium carbonate, sodium or potassium bicarbonate, preferably sodium bicarbonate; in the presence of an organic solvent, typically a C₃-C₈ ketone for example methylisobutylketone; an aliphatic cyclic C₅-C₁₀ hydrocarbon, for example cyclohexane; a C₁-C₆ alkyl ester, for example methyl, ethyl or isopropyl acetate; or an aromatic hydrocarbon for example toluene; preferably in the presence of ethyl acetate or toluene.

(R) —N-propargyl-1-aminoindane (Rasagiline free base) can be obtained by separating the organic phase from the aqueous phase and evaporating off the solvent from the organic phase, preferably under vacuum.

A salt of (R)—N-propargyl-1-aminoindane is preferably a pharmaceutically acceptable salt with an inorganic or an organic acid, typically hydrochloric or metansulphonic acid. Preferably such salt is (R)—N-propargyl-1-aminoindane mesylate (Rasagiline mesylate).

(R) —N-propargyl-1-aminoindane tartrate can be directly converted into (R)—N-propargyl-1-aminoindane mesylate, according to known methods. Alternatively, such conversion can be carried out by unblocking in situ (R)—N-propargyl-1-aminoindane from the tartrate salt, by using an aqueous solution of a base or of a basic salt of an inorganic acid with an alkaline metal, as defined above, to obtain (R)—N-propargyl-1-aminoindane and extracting it in an aprotic apolar solvent as reported above. The organic phase is then separated from the aqueous one and concentrated, for example, under vacuum and then diluted with a C₁-C₄ alkanol, preferably isopropanol. Such organic phase is then distilled off, for example under vacuum, and the residue is diluted with isopropanol at a temperature of about 40-50° C. The mesylate salt is obtained by adding metansulphonic acid into the solution, preferably at 96-99% and heating the mixture for example at reflux temperature.

(R) —N-propargyl-1-aminoindane mesylate (Rasagiline mesylate) in crystalline form can thus be obtained by crystallization from the isopropanol solution, cooling it at a temperature preferably ranging from −20° C. to 40° C., more preferably between about 0° C. and 10° C.

According to a preferred embodiment of the invention, Rasagiline mesylate in crystalline form can be obtained by rapid cooling the isopropanol solution, for example in about 30 minutes, between about 0° C. and 10° C., to obtain a precipitate of Rasagiline mesylate. After that, the crystalline dispersion is heated for about 15-30 minutes, at about 70-75° C., till the almost complete re-dissolution of the precipitate, and finally cooled between about −20° C. and 40° C., preferably at about 0° C. and 10° C., to obtain a precipitate of Rasagiline mesylate.

The crystalline product can be recovered according to known techniques, for example by filtration or by centrifugation, optionally followed by drying in constant-temperature oven, preferably at about 40-70° C., or by drying under vacuum. Preferably the product is recovered by filtration on gooch filter followed by oven drying at about 60° C.

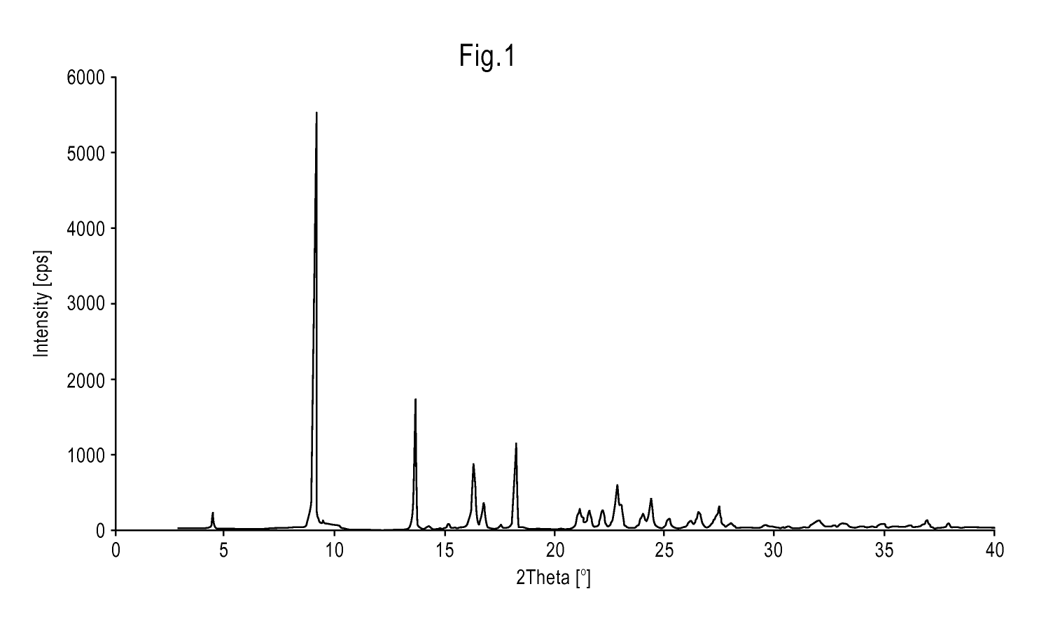

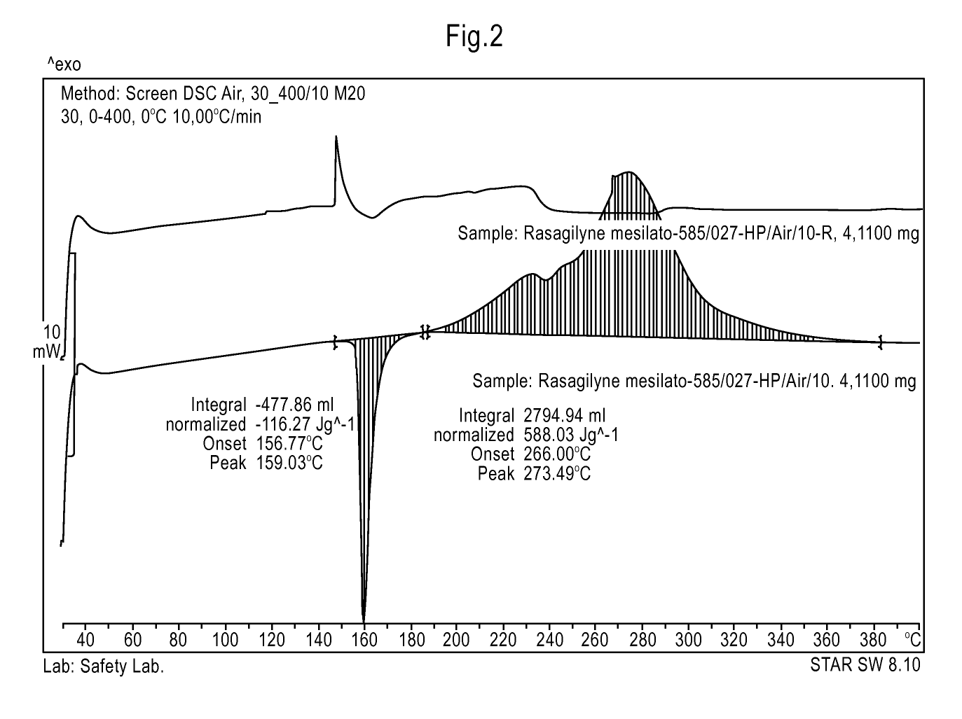

Accordingly, a further object of this invention is Rasagiline mesylate in crystalline form, thus obtainable, having a DSC thermogram as reported in FIG. 2, with an endothermic peak at about 156-157° C.; and a XRPD spectrum as reported in FIG. 1, wherein the most intense diffraction peaks fall at 4.6; 9.1; 13.7; 16.4; 16.8; 18.3; 21.2; 21.7; 22.3; 22.9; 24.4; 26.2; and 27.5±0.2° in 2θ.

The particle size of the crystals of crystalline Rasagiline mesylate, as obtainable according to the invention, is characterized by a D₅₀ value comprised between about 25 and 250 μm, wherein D₅₀ refers to the particle diameter so as to 50% (in volume) of the sample of particles has a diameter equal to or lower than the specific value. Such value, if desired, can be reduced by micronization or fine grinding.

Rasagiline mesylate as obtainable by the process of the invention has a purity equal to or higher than 99.5%, more preferably equal to or higher than 99.9% as determined by HPLC assay.

A further object of this invention is a pharmaceutical composition, particularly in the solid form, for example as tablets, capsules or granulates, comprising as active ingredient crystalline Rasagiline mesylate, as obtainable by the process of the invention, and a suitable carrier and/or excipient. Such pharmaceutical form can be prepared according to known methods.

If desired, (R)—N-propargyl-1-aminoindane (Rasagiline free base) can be obtained in substantially anhydrous crystalline form, herein defined as Form A.

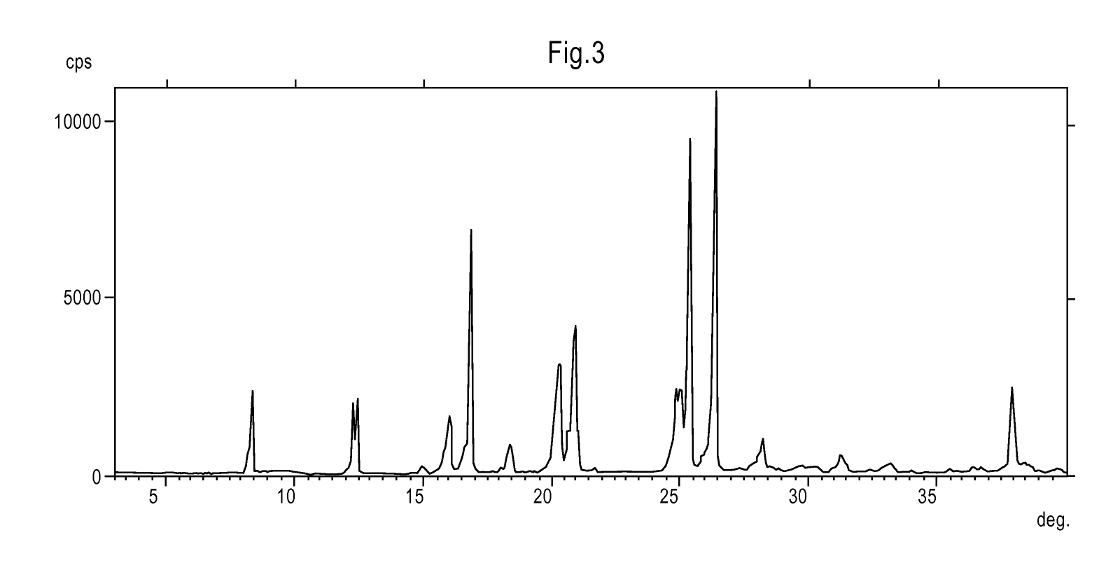

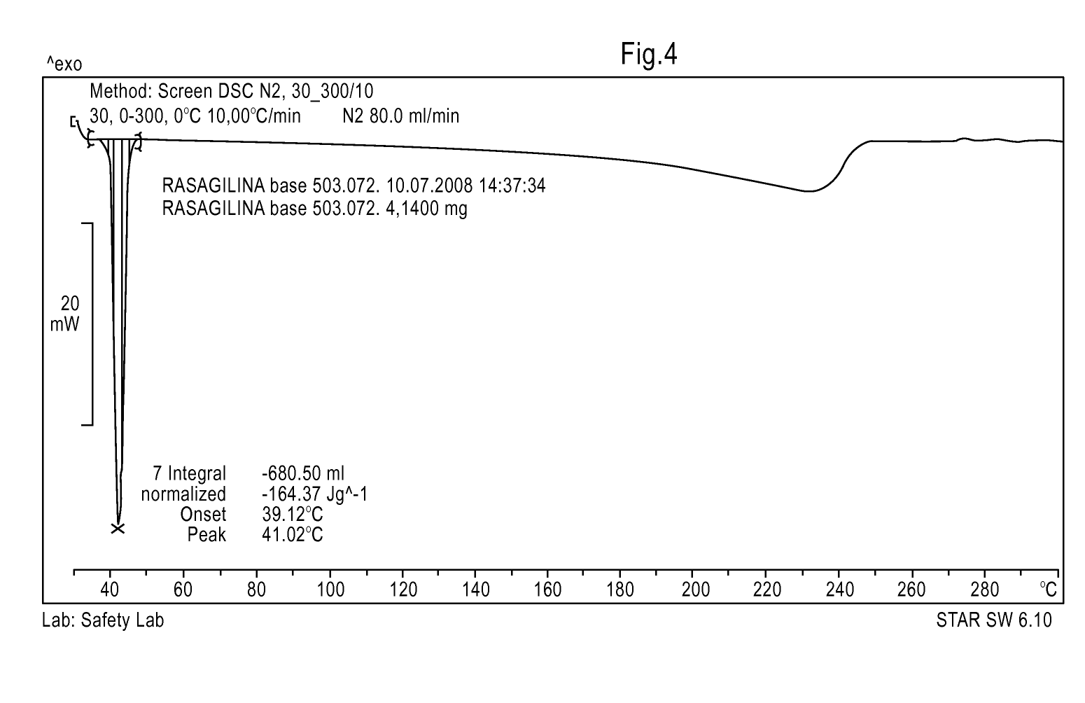

Crystalline Form A has a water content comprised between about 0 and 0.2%, therefore it can be defined as substantially anhydrous. Furthermore, it has a DSC thermogram as reported in FIG. 4, having a melting point comprised between about 39 and 42° C., and a XRPD spectrum as reported in FIG. 3, wherein the most intense diffraction peaks fall at 8.4; 12.3; 12.4; 16.0; 16.8; 20.2; 20.9; 24.9; 25.4 and 26.3±0.2° in 2θ.

According to a further object of the invention (R)—N-propargyl-1-aminoindane as crystalline Form A, as defined above, can be prepared for example by a process comprising:

A1) forming a solution of Rasagiline free base in an organic solvent;
A2) cooling the solution to obtain a precipitate; and
A3) recovering the solid thus obtained; or
B1) forming a solution of Rasagiline free base in an organic solvent;
B2) adding an anti-solvent to the solution to obtain a precipitate; and
B3) recovering the solid thus obtained; or:
C1) forming a mixture of an addition salt of Rasagiline with water and an organic solvent;
C2) treating the mixture with an alkaline metal or alkaline-earth metal base;
C3) separating the phases and concentrating the organic solution till residue;
C4) crystallizing the so obtained residue from an aprotic apolar solvent; and
C5) recovering the so obtained solid.

Rasagiline free base can be prepared for example according to U.S. Pat. No. 5,532,415 or according to the new process disclosed above.

According to variant A1), an organic solvent can be, for example, an aprotic apolar solvent such as a linear or branched, cyclic or polycyclic C₅-C₁₂ alkane, for example hexane, heptane, cyclohexane or decaline, or an aromatic hydrocarbon, for example benzene, toluene or xylene. Preferably it is an apolar aprotic solvent, more preferably a C₅-C₁₂ alkane, in particular cyclohexane.

According to variant A1), the formation of a solution of Rasagiline free base in an organic solvent can be obtained, for example, by heating the dispersion of Rasagiline free base in the solvent at a temperature approximately comprised between 20 and 100° C., preferably between about 40° C. and 85° C.

The concentration of Rasagiline free base in the organic solvent, in particular in an apolar aprotic solvent, as defined above, is typically comprised between 0.1M and 10M, preferably between 1M and 2M.

According to variant A2) the cooling of the solution, to obtain a precipitate of solid Rasagiline, can be carried out at a temperature typically comprised between about −20° C. and 40° C., preferably approximately between 0° C. and 10° C.

The crystalline solid product can be recovered according to known techniques, for example by filtration or centrifugation, optionally followed by drying under vacuum. Preferably the product is recovered by filtration followed by drying under vacuum at room temperature.

According to variant B1) an organic solvent can be for example, an aprotic polar solvent, typically dimethylformamide, dimethylacetamide, aceto nitrile, dimethylsulfoxide; an ether for example diethylether, methyl-tert-butylether, tetrahydrofuran or dioxane; a chlorinated solvent, for example, dichloromethane, dichloroethane, chloroform o chlorobenzene; an apolar aprotic solvent, such as an aromatic hydrocarbon, for example benzene, toluene o xylene; an ester, for example ethyl or methyl acetate; or a C₃-C₁₂ ketone, for example acetone, methylethylketone, methylisobutylketone; or a mixture of two or more, preferably two or three of said solvents. Preferably it is selected among acetonitrile, ethyl acetate and tetrahydrofuran, in particular it is ethyl acetate.

According to variant B1) a solution of Rasagiline free base in an organic solvent can be obtained for example as disclosed above for process variant A1). Analogously, the concentration of Rasagiline free base in the organic solvent, in particular in an aprotic polar solvent, is typically comprised between 1M and 10M, preferably between 2M and 5M.

According to variant B2), an anti-solvent is for example an apolar aprotic solvent, preferably a linear or branched, cyclic or polycyclic C₅-C₁₂ alkane, more preferably hexane, heptane, cyclohexane or decaline.

The recovering of the solid can be obtained for example as disclosed above for process variant A3).

An addition salt of rasagiline Rasagiline can be, for example, a pharmaceutically acceptable acid addition salt, for example a salt with an inorganic strong acid or an organic carboxilic or sulphonic acid, typically hydrochloric, tartaric or methansulphonic acid. Preferably such salt is Rasagiline mesylate.

According to variant C1), an organic solvent can be any solvent which can dissolve Rasagiline free base which forms in the subsequent step C2), such as a polar or apolar, protic or aprotic solvent as defined above, preferably methylene chloride, toluene, chloroform, acetone, tetrahydrofuran, ethyl acetate and acetonitrile, more preferably ethyl acetate.

A base of an alkaline or alkaline-earth metal can be for example an inorganic base such as a hydroxide of an alkaline or of an alkaline-earth metal such as sodium, potassium or barium hydroxide; or a basic salt with an inorganic acid such as a carbonate or bicarbonate of an alkaline or alkaline earth metal, typically sodium bicarbonate or potassium carbonate, preferably sodium bicarbonate.

The concentration to residue of the organic solution can be carried out according to known methods.

The crystallization of the so obtained residue can be carried out, first by dissolving the residue in an apolar aprotic solvent, as defined above, preferably a linear or branched, cyclic or polycyclic C₅-C₁₂ alkane, more preferably cyclohexane and, if necessary, heating the so formed mixture, preferably in a range comprised between about 25° C. and 85° C.; and then by cooling the solution to obtain a precipitate of Rasagiline, preferably at a temperature typically comprised between −20° C. and 40° C., more preferably approximately between 0° C. and 110° C.

The subsequent recovery of Rasagiline free base in crystalline form can be carried out as disclosed above.

The dimensions of the crystals of crystalline solid Rasagiline free base, obtained according to the methods herein disclosed, is characterized by a D₅₀ value comprised between about 25 and 250 μm, wherein D₅₀ is the particle diameter so as to 50% (in volume) of the particle sample has a diameter equal to or lower than the specific value. Such value, if desired can be reduced by micronization or fine grinding. Rasagiline crystalline free base has a purity equal to or higher than 99.5%, preferably equal to or higher than 99.9%, as by HPLC assay.

Due to the high purity of Rasagiline free base, as obtainable by the process of the invention, it can be advantageously used in the preparation of the salts of Rasagiline, for example the mesylate salt, having high purity, for example equal to or higher than 99.5%, preferably equal to or higher than 99.9%, as by HPLC assay.

A further object of this invention is a pharmaceutical composition, in particular in the form of a medicated patch, preferably a transdermal patch, comprising as active ingredient Rasagiline free base in solid form, in particular in substantially anhydrous form, preferably as Form A, as herein defined, and a suitable carrier and/or excipient. Such pharmaceutical formulation can be prepared according to known methods.

The following examples illustrate the invention.

### Example 1

### Preparation of N-propargyl-1-aminoindane

In a IL rector, maintained under nitrogen atmosphere, sodium borohydride (22.9 g; 605.34 mmol) and tetrahydrofuran (320 mL) are added. The suspension is cooled at 0-5° C., and under stirring glacial acetic acetic (116.5 g; 1940.05 mmol) is added in 1.5 hours, keeping the temperature under 15° C. The suspension is then heated to about 20-25° C. and stirred for about 20 minutes. The reaction mixture is heated to about 30-35° C. and 1-indanone (40 g; 320.66 mmol) is added. The mixture is then stirred for 5-10 minutes and propargylamine (42.7 g; 757.08 mmol) is dropped, in at least three hours. The mixture is maintained under stirring to complete reaction. The mixture is then cooled to 20-25° C. and water (220 mL) is added. Potassium carbonate is added till the pH remains between 7 and 8. The mixture is heated, keeping it under stirring for about 15 minutes and the phases separates. The organic phase is distilled off to residue under vacuum and 47.43 g of N-propargyl-1-aminoindane are obtained. Yield 91%.

### Example 2

### Preparation of (R)—N-propargyl-1-aminoindane Mesylate

In a IL rector maintained under nitrogen atmosphere, N-propargyl-1-aminoindane (47.43 g; 277 mmol) obtained from Example 1, ethanol (340 mL) and L(+)-tartaric acid (21.2 g; 141.25 mmol) are added. The mixture is refluxed for about 1 h. Then the mixture is cooled at 0-5° C., in 5-6 h, and kept to such temperature for about 1 h. The mixture is filtered and the filter washed with 0-5° C. pre-cooled ethanol. 48 g of wet solid are obtained which are dried in oven at 60° C. to constant weight. 31.6 g of (R)—N-propargyl-1-aminoindane tartrate are obtained.

(R) —N-propargyl-1-aminoindane tartrate (31.6 g; 128.46 mmol), thus obtained, is loaded in a 1 L reactor and maintained under nitrogen atmosphere. Ethyl acetate (217 mL), sodium bicarbonate (13.5 g; 160.71 mmol) and water (190 mL) are added. The mixture is stirred to complete dissolution at 20-25° C. The phases are separated and the organic one is washed with water (30 mL). The phases are separated again and the organic one is concentrated under vacuum, obtaining (R)—N-propargyl-1-aminoindane as an oily residue. The residue is taken up with isopropanol (50 mL) and the solvent distilled off under vacuum to dryness. The residue is then cooled at 40-50° C. and taken up with isopropanol (65 mL). Metansulphonic acid (11.5 g; 119.66 mmol) is added to the solution. The mixture is heated to reflux to obtain a solution which is discoloured by adding carbon. The clear solution is cooled at 75° C., and left to crystallize. The solution is cooled at 0-5° C. in 5-6 h and maintained at such temperature for at least 1 h. 32 g of wet solid are recovered by filtration, the solid is dried in oven at 60° C. to constant weight. 28.7 g of (R)—N-propargyl-1-aminoindane mesylate are obtained. Yield: 89% from (R)—N-propargyl-1-aminoindane tartrate. The product has a DSC thermogram as reported in FIG. 2, and a XRPD spectrum as illustrated in FIG. 1, wherein the most intense diffraction peaks fall at 4.6; 9.1; 13.7; 16.4; 16.8; 18.3; 21.2; 21.7; 22.3; 22.9; 24.4; 26.2; and 27.5±0.2° in 2θ.

### Example 3

### Preparation of (R)—N-propargyl-1-aminoindane Mesylate

By proceeding according to Example 2, the isopropanol solution of Rasagiline mesylate, which is obtained after decoloration by adding carbon, is quickly cooled for example in about 30 minutes, at about 0° C. and 10° C., to obtain a precipitate of Rasagiline mesylate. Subsequently the crystalline dispersion is heated for about 15-30 minutes, to about 70-75° C., to almost complete redissolution of the precipitate and finally cooled to about −20° C. and 40° C., more preferably to about 0° C. and 10° C., to obtain a precipitate of crystalline Rasagiline mesylate. The product has a DSC thermogram as reported in FIG. 2 and a XRPD spectrum as illustrated in FIG. 1, wherein the most intense diffraction peaks fall at 4.6; 9.1; 13.7; 16.4; 16.8; 18.3; 21.2; 21.7; 22.3; 22.9; 24.4; 26.2; e 27.5±0.2° in 2θ.

### Example 4

### Preparation of Crystalline Form of (R)—N-propargyl-1-aminoindane Free Base

(R)-Rasagiline oil (10.1 g, 38 mmoli), obtained according to Example 2, is suspended in a mixture of water (50 ml) and ethyl acetate (200 ml) and treated with a saturated solution of sodium bicarbonate to a pH range comprised between 8 and 9. The phases are separated and the organic one is washed with water (2×50 ml) and filtered on paper, then it is concentrated under reduced pressure to constant weight. The so obtained oily residue is treated with cyclohexane (25 ml) and the so obtained mixture is heated under stirring till a clear solution is obtained. The solution is then slowly cooled and maintained under stirring for about 3 hours at 5° C. Crystals form which are filtered and washed with cyclohexane and dried under vacuum. 5.1 g of Rasagiline free base are obtained in crystalline solid form, with a yield of 78%; m.p. 40-41° C.; and HPLC purity of 99.8%. The water content in the compound determined by titration according to Karl Fischer is about 0.1%. The product has a DSC thermogram as reported in FIG. 4, and an XRPD spectrum as shown in FIG. 3, wherein the most intense diffraction peaks fall at 8.4; 12.3; 12.4; 16.0; 16.8; 20.2; 20.9; 24.9; 25.4 e 26.3±0.2° in 2θ.

1. A process for the preparation of N-propargyl-1-aminoindane comprising reacting 1-indanone of formula (II) 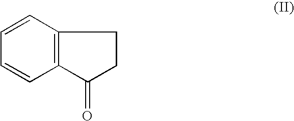
with propargylamine in the presence of a mixture of sodium borohydride and acetic acid.

2. Process according to claim 1, wherein the acetic acid is glacial acetic acid.

3. Process according to claim 1, wherein the reaction is carried out in a solvent selected from the group consisting of an aromatic hydrocarbon, a cyclic ether and an acyclic ether.

4. Process according to claim 3 wherein the solvent is tetrahydrofuran.

5. Process according to claim 1 wherein the reaction is carried out at a temperature ranging from about 10 to about 40° C.

6. Process according to claim 5 wherein the reaction is carried out at a temperature of about 30° C.

7. Process according to claim 1, further comprising the conversion of N-propargyl-1-aminoindane into (R)—N-propargyl-1-aminoindane or a salt thereof.

8. Process according to claim 7 wherein the salt is the mesylate salt.

9. Crystalline form of (R)—N-propargyl-1-aminoindane mesylate, having a XPRD spectrum as shown in FIG. 1 wherein the most intense diffraction peaks fall at 4.6; 9.1; 13.7; 16.4; 16.8; 18.3; 21.2; 21.7; 22.3; 22.9; 24.4; 26.2; and 27.5±0.20 in 2θ.

10. Process for the preparation of crystalline form of (R)—N-propargyl-1 aminoindane mesylate, as defined in claim 9, comprising:
quick cooling of an isopropanol solution of (R)—N-propargyl-1 aminoindane mesylate to approximately 0-10° C., to obtain a precipitate of (R)—N-propargyl-1-aminoindane mesylate;
heating the crystalline dispersion to almost complete dissolution of the precipitate;
cooling the dispersion to a temperature comprised approximately between −20 and 40° C.; and
recovering the crystalline product.

11. A process for the preparation of (R)—N-propargyl-1-aminoindane having a XRPD spectrum with the most intense diffraction peaks falling at 8.4; 12.3; 12.4; 16.0; 16.8; 20.2; 20.9; 24.9; 25.4 and 26.3±0.2° in 2θ, comprising:
A1) providing a solution of rasagiline base in an organic solvent;
A2) cooling the solution to obtain a precipitate; and
A3) collecting the solid;
or
B1) providing a solution of rasagiline base in an organic solvent;
B2) adding an anti-solvent to the solution to obtain a precipitate; and
B3) collecting the solid
or
C1) forming a mixture of an addition salt of Rasagiline with water and an organic solvent;
C2) treating the mixture with an alkaline metal or alkaline-earth metal base;
C3) separating the phases and concentrating the organic solution to residue;
C4) crystallizing the so obtained residue from an aprotic apolar solvent; and
C5) recovering the solid.

12. A process according to claim 11 wherein in variant A1) the organic solvent is an aprotic apolar solvent comprising a linear or branched, cyclic or polycyclic C₅-C₁₂ alkane, or an aromatic hydrocarbon.

13. A process according to claim 11 wherein in variant B1) an, the organic solvent is an aprotic polar solvent, comprising dimethylformamide, dimethylacetamide, aceto nitrile, or dimethylsulfoxide or an ether or a chlorinated solvent or an apolar aprotic solvent comprising an aromatic hydrocarbon, an ester or a C₃-C₁₂ ketone or a mixture of two or more of said solvents.

14. A process according to claim 11 wherein in variant C1) the organic solvent methylene chloride, toluene, chloroform, acetone, tetrahydrofuran, ethyl acetate or acetonitrile, preferably ethyl acetate.

15. A process according to claim 11 wherein in variant C4) the aprotic apolar solvent is a linear or branched, cyclic or polycyclic C₅-C₁₂ alkane, preferably cyclohexane.

16. Crystalline form of (R)—N-propargyl-1-aminoindane mesylate, wherein the compound has a DSC thermogram as shown in FIG. 2 with an endothermic peak at about 156-157° C.

17. Crystalline form of (R)—N-propargyl-1-aminoindane mesylate, wherein the crystals have a D₅₀ value of about 25 to 250 μm.

18. Process of claim 8, wherein the mesylate salt has a purity of at least 99.5% as determined by HPLC assay.

19. Process of claim 18, wherein the purity is at least 99.9%.
